(19) United States
(12) Patent Application Publication
(10) Pub. No.: US 2010/0145262 A1
(43) Pub. Date: Jun. 10, 2010

(54) SAFETY SYSTEM FOR INSULIN DELIVERY ADVISORY ALGORITHMS

(75) Inventors: Henrik Bengtsson, Taastrup (DK); Leif Engmann Kristensen, Hoersholm (DK); Joergen Smedegaard, Vanloese (DK); Morten Simoni Spjuth, Aalborg (DK); Ole Skyggebjerg, Hilleroed (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/598,283

(22) PCT Filed: Apr. 7, 2008
(86) PCT No.: PCT/EP2008/054149

§ 371 Date: Feb. 26, 2010

(30) Foreign Application Priority Data
May 3, 2007 (EP) 07107411.6

(51) Int. Cl. A61M 5/168; G05B 19/00

(52) U.S. Cl. 604/66; 340/5.2

(57) ABSTRACT

A closed loop control system for a medical drug delivery device logs historical data such as drug delivered and corresponding physiological parameters such as blood glucose level. On the basis of the logged historical data, the system calculates an estimated event forecast on which basis the system further calculates the necessary, prudent future drug delivery profile in order to counter delay in the closed loop. The estimated event forecast is presented to the user, the user is thereby given the possibility to accept, reject or adjust the event forecast and the corresponding drug delivery profile. Dynamic and adaptive safety limits to the drug delivery and the physiological parameter can be set by the user and will dynamically follow the drug delivery profile and the event forecast and will further optimize based on the learned behavioural pattern of the user. The user can set the safety level as a percentage of deviation to the drug delivery profile.

### TECHNICAL FIELD

The present invention relates to advisory control systems for insulin delivery systems. Despite the ongoing development and refinement of control systems or the like systems for advising and possibly regulating delivery of insulin to a user suffering from diabetes, these has difficulties in obtaining perfect trim of the patients' blood glucose level because of a number of physiological delaying factors. The present invention deals with this problem by presenting an intelligent and adaptive safety system as well as a new event predicting function which improves the regulation of insulin delivery and therefore the patients' blood glucose profile. The systems of the present invention may be provided as portable devices and may for example be included in a medication delivery apparatus.

### BACKGROUND OF THE INVENTION

Treatment of diabetes aims at keeping the blood glucose level of the patient (hereafter referred to as the user) as close to the glucose level of a comparable, non-diabetic individual. This is desired in order to minimize the short term and long term risks which are the consequence of blood glucose levels different from the normal.

To obtain a well balanced treatment it is an advantage to perform many discrete insulin deliveries to the body of the user, thereby approximating a continuous insulin flow—approximating the natural insulin delivery in the body of a comparable, non-diabetic individual. Evidently such treatment is difficult to obtain when treating diabetes with insulin injected by a syringe, as this can not be done in a repeatedly daily number without considerable discomfort to the user. Therefore insulin pump technology is desirable, as insulin pumps is able to deliver insulin in a large number of daily doses, where the dose size is varied according to the requirement of the user, thus pump therapy approximates a continuous insulin flow.

Insulin pumps therefore remove the main part of problems and discomfort relating to the actual delivery of the insulin, once the pump is in place. However, the main focus then shifts from the delivery of the insulin from the pump to controlling the pump in accordance to the requirements of the user. An example of an insulin pump is described in U.S. Pat. No. 6,558,351, which discloses a method of controlling an insulin pump via feed-back from a continuous blood glucose meter (CGM) in a closed loop system. Closed loop system for diabetes treatments will in the coming future become possible and useable after continuous blood glucose meters (CGM) has entered the marked.

Based on the new CGM devices a number of closed loop clinical trails have been performed. Most of these are using a simple human model to predict future blood glucose (BG) levels from the insulin flow-rate set by the closed loop control system. This predicting makes it possible for the closed loop system to have a good estimate of the effect when setting the dose, in stead of having to wait for the pharma-dynamic reaction on BG.

Another approach that has been tried out with equal good results is using a simple PID-control (known from basic motor control systems). The PID controller is able to handle most situations well but has problems with meals and other fast acting disturbances.

However good the control algorithm, the control system of an insulin pump, as described in the art as for instance in U.S. Pat. No. 6,558,351, this control is complicated by the fact that when administering insulin through the skin of a user, at least three different delay factors are present and physiologically unavoidable, complicating the control:
There is a pharma kinetic delay from the insulin is delivered to the user until it is present in the bloodstream, the length of this delay varying depending on the insertion or delivery technique;
there is a pharmadynamic delay which is the delay from the moment the insulin is present in the bloodstream until the body reacts to the insulin;
and there is the delay from the moment the body reacts to the insulin until the blood glucose level in the body stabilises so a true blood glucose level can be measured via a CGM-sensor (Continuous Glucose Monitoring) to constitute as a feed-back to the insulin pump controller, including the delay of the device analyzing the blood in order to achieve a glucose value.

These delays can range from approximately 50 minutes to approximately 90 minutes, and hence prevent effective compensation of large disturbances of the endogenous glucose balance introduced by large carbohydrate intake or exercise. Accordingly, all present insulin pump delivery control systems have the problem that many fast acting disturbances such as meals and exercise can change the blood glucose level or the blood glucose level profile well before the insulin pump controller is able to react. The pump is always being controlled on the basis of historical (old) and potential false, misleading data, as the reality of the users condition can well have changed within the delay time-window. In addition the input to the closed-loop may be erroneous and misleading, e.g. caused by an erroneous sensing device.

However good the insulin pump control system is, this time delay is an unavoidable fact and problem, and therefore there is a need for a safety system. A safety system is further needed to counter the limits and delays in the pump control system, the safety system can be habit learning.

### SUMMARY OF THE INVENTION

It is an object of the present invention to provide a system capable of ensuring that a closed loop insulin delivery system will function safely despite the closed loop limitations by introducing adaptive safety limits and prediction of future disturbances based on historical data and optionally user acknowledgements and user input.

The present invention provides a system for logging historical data concerning a user's behavioural patterns, insulin delivery profile (to the user), his/her blood glucose profile and possibly further physiological data. The system comprises processing means for calculating a near-future desired and prudent insulin delivery profile based on these logged data and also based on expected future critical disturbances influencing the insulin demand (mainly food intake and exercise as mentioned before as well as sleep). This calculation is done on the basis of the “experience” learned from the logged historical data. The control system seeks to discover a main trend in the historical data. For a user who has a stable daily or weekly rhythm of food intake size, food intake time of day, wake-up time, bed time, pattern of exercise and amount of exercise etc., the control system will then be able to estimate a close to true behavioural pattern and on this basis further calculate the near-future desired and prudent insulin delivery profile. Via output means comprised in the system, this calculated insulin delivery profile can be communicated from the control system to the insulin pump. Further, a man-machine-interface (MMI) such as a display and input means such as push buttons, scroll wheels, touch pads, touch sensitive screens and the like enables interaction between the user and the control system. The control system can present predicted critical disturbances, an “event forecast” to the user, based on the logged historical data. In this manner the user does not need to be pro-active and remember to input future critical disturbances to the control system which not only entails encumbrance to the user, but more seriously implies the risk that the user may forget to input the critical disturbance in due time. On the contrary, the control system of the present invention is pro-active, and presents the expected critical disturbance in due time, where “in due time” in this context means in time for the control system to send the related correct output to the insulin pump considering the vast time-delay as described in the foregoing. Hence, the user needs only to accept the event forecast proposed by the control system, and the system will subsequently calculate a prudent insulin delivery profile accordingly, for the insulin pump to follow. If the event forecast is not in accordance with the actual planned near-future activities of the user, he/she has the choice of delaying the event, adjust the event time, length, size, intensity or the like, cancelling the event or inputting an alternative event. This last mentioned feature of the system is especially important for a user who does not live his/her life according to a stable daily or weekly pattern, hence, a habit-tracking/-detection is performed and this information is used prospective. Normally in the state of the art, the best insulin treatment is achieved when the user lives a stable life, since it is quite difficult to calculate appropriate insulin delivery (time and dose size) when changes in the users “normal” life pattern occurs. But in the present system, it is still possible to achieve a near to normal blood glucose level profile of the user even when he lives a life with critical events occurring outside the “normal” life pattern, as long as the user inputs the critical events to the control system, preferably in advance of the event occurring The input and output of commands can be performed in any state of the art manner, such as text, symbols, colour, sound, vibration, speech, this list not being exhaustive. The user can at any time manually input an event not foreseen by the predictive system, which enables the control system to adjust the insulin delivery profile accordingly as fast as possible with respect to the time delay factors. It is a further feature of the invention that the user can input a variable default accept threshold whereby event forecasts and their corresponding infusion rate profiles lying within the user input accept threshold are implemented in the control of the infusion device without specific user acceptance of the actual event. This means that as the user's confidence in the control systems adequate and prudent control of the users' blood glucose level ascends, the user can surrender an ascending level of control to the system. For instance, the user can set a default threshold value which lets the system control the expected change in insulin profile according to the time of day and the daily lunch meal and sleep time without asking the user for accept of the occurrence of these events.

Though the systems pro-active event forecast and user accept feature as described in the foregoing effectively minimizes the risk of misestimating the prudent near-future insulin delivery profile, there is still a risk that the user may perform an action or be exposed to activities not foreseen by the control system, which may have a critical effect on the blood glucose level. Therefore the system according to the present invention also comprises a safety system able to perform a warning and able to perform preventive actions.

It is already known in the art to equip the control system with safety limits, but the present invention has a clear advantage over the known art, since the safety limits of the present invention are adaptive, as will be explained in the following.

Though insulin is an essential necessity for the body, it is also a potential thread to the body, if delivered in the wrong doses. Therefore it is known to equip insulin pump control systems with insulin dose safety limits defined by the user or a health care person. However the problem is that static safety limits can be too high in some situations, where the user only needs small doses of insulin, for instance due to exercise, and in other situations the safety limits can be too low in situations where the user needs large doses of insulin, for instance due to feed intake. The static safety limits therefore need to be set conservatively which leads to many unwished false alarms. The false alarms are cumbersome to the user, who must in every case relate to the alarm and evaluate the current situation, but more seriously, the users trust in the control system is affected negatively. The present invention solves this problem by taking advantage of control systems ability to log historical data including the insulin delivery profile related to events in the user's behavioural pattern. On this basis the control system calculates dynamic insulin delivery safety limits, which move up and down over time relating to the body's required prudent insulin delivery profile. The user or health care person then only input safety limits defined as an area around the required prudent insulin delivery profile, which varies depending on the time of day and the user activity throughout the day, that is, for instance the safety limits can be plus or minus 5% of the at any time required insulin delivery profile. When the user or health care person has gained more trust in the control system, based on the experiences, the safety limits can be expanded (8%, 10% etc.), thereby enabling it to act between wider limits and minimizing the number of alarms or preventive actions from the control system. The safety limits can be divided into levels with different actions allied, such as a “green light zone” in which the insulin profile is according to the estimated, a “yellow light zone” where the user is alerted and a “red light zone”, where the control system shifts from automatic control to manual control or to stop of insulin delivery. As the control loop includes a CGM as mentioned earlier, the adaptive safety limits can besides the insulin delivery profile also relate to the blood glucose level. Again, the safety limits for the blood glucose level are then adaptive relating to the actual situation and activity of the user that is the safety limits can dynamically change over time relating to the situation of the user, the systems event forecast and the calculated behavioural pattern of the user. The safety limits for insulin as well as blood glucose need not be symmetrically placed around the insulin profile/blood glucose profile. There can be a higher or lower span from the profile to the upper limit as compared to the lower limit. For instance it could be accepted to have a higher span from the insulin profile to the lower limit than to the upper limit, whereas it can be advantageous to accept a higher span from the optimal blood glucose profile to the upper limit than to the lower limit in order to reduce the risk of a hypoglycaemic episode of the user. Further the profile of the upper limit need not be identical to the profile of the lower limit. This can be particularly relevant when concerning the blood glucose level profile, since it might be accepted for shorter periods to have high blood glucose levels, for instance if it is expected that high physical activity is imminent, while it might never be accepted to have blood glucose levels below a certain fixed level. In this case it might be the case that the upper safety limit is dynamically changing over time, but the lower limit is a constant fixed set value, or both the upper and lower safety are dynamically changing, but the lower safety limit has a further constant fixed set value which is superimposing the lower dynamic safety limit.

### FEATURES OF THE INVENTION

1. A partial or fully closed loop infusion control system based on model prediction, a constraint anticipatory model predictive controller, a model predictive controller, a LQG controller using kalman filter or extended kalman filter and state-of-the-art system identifications techniques for controlling the infusion of a medical drug into a user characterised in
sensing means for sensing of at least one physiological parameter, storing means for storing the at least one physiological parameter and infusion rate over time and occurrences of events as historical data,
processing means for adaptation to the individual by use of advanced system identifications methods and further to identify behavioural pattern of the user based on said historical data,
output means for generating an output, said output represents a drug infusion profile, audio and/or visual display means for presenting an output to the user, input means enabling the user to perform manual input to the control system and an infusion device in communication with said closed loop infusion control system, characterised in that
MMI processing means via the display means presents an event forecast and a reminder to the user based on said generated behavioural pattern, said event forecast corresponds a drug infusion rate profile which initiates on the precondition that the user accepts the event forecast.

2. A closed loop infusion control system according to feature 1 characterised in that —

said physiological parameter is the users blood glucose level or interstitial glucose level or glucose level in cutis, measured discrete or continuous for partial or fully closed loop insulin administration purpose.

3. A closed loop infusion control system according to feature 1 or 2 characterised in that—

the control system comprises a variable user input default accept threshold whereby event forecasts and their corresponding infusion rate profiles lying within said user input accept threshold are implemented in the control of the infusion device without explicit user acceptance of the actual event.

4. A closed loop infusion control system according to any of the preceding features characterised in that—the control system comprises dynamic safety limits or constraints for insulin delivery to the user, whereby said safety limits dynamically surrounds the expected insulin flow based on the model prediction and/or the average insulin profile based on historical data. The safety limits are dynamic and follows rises and falls of the expected insulin flow, said safety limits thereby has a user defined distance to the insulin profile

5. A closed loop infusion control system according to any of the preceding features characterised in that—

the control system comprises dynamic safety limits for the glucose level of the user, whereby said safety limits dynamically surrounds the profile of the glucose level as the insulin infusion level rises and falls, said safety limits thereby has a user defined distance to the glucose level.

6. A closed loop infusion control system according to any of the preceding features characterised in that—the control system comprises dynamic safety limits (constraints for insulin delivery to the user), whereby said safety limits dynamically surrounds the model predictive or average insulin profile based on historical data. The safety limits are dynamic according to the model predictive dataset (estimation error, insulin data, glucose data, event data, meal, activity etc.) insulin infusion level rises and falls, said safety limits thereby has a user defined distance to the insulin profile

7. A closed loop infusion control system according to feature 4-6 characterised in that—

the upper dynamic safety limit is not symmetrically displaced above the insulin profile/the glucose level as compared to the lower dynamic safety limit.

8. A closed loop infusion control system according to feature 4 to 7 characterised in that—

at least one static user set limit correlates the dynamic limit, said static limit is independent of the actual insulin or blood glucose level.

9. A closed loop infusion control system according to any of the preceding features characterised in that—

said event forecast and said corresponding drug infusion rate profile is adaptive, whereby it over time learns to estimate increasingly accurate events and corresponding drug infusion rate profiles on the basis of the logged historical data, as the base of historical data increases.

10. A closed loop infusion control system according to any of the preceding features characterised in that—

the user has the possibility of including or excluding an occurring event from the historical data storage.

11. A closed loop infusion control system according to any of the preceding features characterised in that—

the model predictive result and the measured data is compared and a system identification performance parameter for the model is used in the algorithm for further stability and alarm for when the predictive model is of to low quality to rely on, said parameter is used as a measure of the overall control systems adaptation performance and reliability.

12. A closed loop infusion control method based on model prediction said a constraint anticipatory model predictive controller, a model predictive controller, a LQG controller using kalman filter extended kalman filter and state-of-the-art system identifications techniques for controlling the infusion of a medical drug into a user characterised in the following steps —
sensing of at least one physiological parameter, storing means for storing the at least one physiological parameter and infusion rate over time and occurrences of events as historical data,
processing for adaptation to the individual by use of advanced system identifications methods and further to identify behavioural pattern of the user based on said historical data,
generating an output, said output represents a drug infusion profile, audio and/or visual display means for presenting an output to the user, input means enabling the user to perform manual input to the control system and an infusion device in communication with the closed loop infusion control system,
display on a MMI display an event forecast and a reminder to the user based on said generated behavioural pattern, said event forecast corresponds a drug infusion rate profile which initiates on the precondition that the user accepts the event forecast
Introducing one or more safety limits between which the closed loop control system is given user authorization to operate
setting the safety limit(s) by user input and/or by the control system, based on historical insulin infusion data and at least one further parameter.

13. A method according to feature 12 where the at least one further parameter is: physiological interstitial glucose level, system identification performance parameter or event monitoring, and said event is a meal or postprandial sleep or exercise or post-hypo.

14. A method according to feature 12 or 13 where the safety limits can be set in one or more levels, preferable 3 levels: A work area, a warning area, and an alert area where at least one of the safety limits is broken.

15. A method according to any of the features 12-13 where the at least one safety limit is based or partially based on accumulated insulin flow over a specific user defined period.

16. A method according to any of the features 12-15, where a safety measure is taken before the actual safety limit is reached, based on the margin between actual blood glucose level and said safety limit and the blood glucose derivative, said derivative being a steep fall or a steep rice in blood glucose level.

17. A method according to any of the features 12-16, where the at least one safety limit is changed up or down relative to the insulin infusion rate axis or into different event modes, such as high, low, normal and sleeping; based on event monitoring

18. A method according to any of the features 12-17, where resting/sleep is detected preferable by ECG measurement and the boundary limits are optimized on this basis.

19. A method according to any of the features 12-18 where the user can change modes from the control system normal mode to a safe-mode or completely to manual mode for a period of time.

20. A method according to any of the features 12-19 where a detection of hypoglycemia or a retrospective detection of hypoglycemia is used to change the safety limits.

21. A method according to any of the features 12-20 where hypoglycemia is detected retrospectively and provides feedback to the closed loop control system by influencing the safety limits as well as the closed-loop control algorithm.

23. A method for the control system to initially adapt to the individual by use of system identification techniques—based on
user involvement and instructions for the user to perform different metabolic test. (eat 50 g of sugar etc., run 5 km, don't eat the next 10 hours.)
take additional BG-measurements, Calibrate system, go through event-log and clean wrong event estimates.
use additional measurement equipment like activity monitor, in-viro CGM
use input as height, weight, physiological and fitness age, sex, diabetes sub-type and other medical parameters for identification of the optimal model estimation.

101. A medical device control system for controlling the delivery of insulin into a user comprising a blood glucose sensor for sensing at least one physiological parameter, storing means for storing the at least one physiological parameter and delivery rate over time and occurrences of events as historical data, processing means to generate a behavioural pattern for the user based on said historical data, output means for generating an output, said output represents an insulin delivery profile, audio and/or visual display means for presenting an output to the user, input means enabling the user to perform manual input to the control system and a medical device in communication with said control system, said processing means via the display means presents an event forecast and a reminder to the user based on said generated behavioural pattern, said event forecast corresponds an insulin delivery rate profile which initiates on the precondition that the user accepts the event forecast, characterised in that—

the control system comprises dynamic safety limits for the insulin profile of the user, whereby said safety limits dynamically surrounds the insulin profile as the insulin delivery level rises and falls, said safety limits thereby has a user defined distance to the insulin profile.

102. A medical device control system according to feature 101 characterised in that —

said physiological parameter is the users blood glucose level.

103. A medical device control system according to any of the preceding features characterised in that—

the control system comprises dynamic safety limits for the glucose level of the user, whereby said safety limits dynamically surrounds the profile of the glucose level as the insulin delivery level rises and falls, said safety limits thereby has a user defined distance to the glucose level.

104. A medical device control system according to any of the preceding features characterised in that—

the control system comprises a variable user input default accept threshold whereby event forecasts and their corresponding delivery rate profiles lying within said user input accept threshold are implemented in the control of the delivery device without specific user acceptance of the actual event.

105. A medical device control system according to any of the preceding features characterised in that—

the upper dynamic safety limit is not symmetrically displaced above the insulin profile/the glucose level as compared to the lower dynamic safety limit.

106. A medical device control system according to any of the preceding features characterised in that—

at least one static user set limit correlates the dynamic limit, said static limit is independent of the actual insulin or blood glucose level.

107. A medical device control system according to any of the preceding features characterised in that —

said event forecast and said corresponding insulin delivery rate profile is adaptive, whereby it over time learns to estimate increasingly accurate events and corresponding insulin delivery rate profiles on the basis of the logged historical data, as the base of historical data increases.

108. A medical device control system according to any of the preceding features characterised in that—

the user has the possibility of including or excluding an occurring event from the historical data storage.

109. A method for giving user authorization and limiting a medical device control system; the system controlling the delivery of insulin into a user, said system comprising a sensor for sensing one or more physiological parameter(s) of the user, storing means for storing delivery rate over time and at least one physiological parameter as historical data, processing means to generate a behavioral pattern for the user based on said historical data, output means for generating an output, said output represents an insulin delivery profile, audio and/or visual display means for presenting an output to the user, input means enabling the user to perform manual input to the control system and a medical device in communication with said medical device control system, characterized by the steps of:
Introducing one or more safety limits between which the medical device control system is given user authorization to operate.
setting the safety limit(s) by user input and/or by the control system, based on historical insulin delivery data and at least one further parameter,
dynamically changing at least one safety limit up or down relative to the insulin delivery rate axis or into different event modes, such as high, low, normal and sleeping; based on event monitoring.

110. A method according to feature 109 where the at least one further parameter is: physiological interstitial glucose level or event monitoring, and said event is a meal or postprandial sleep or exercise or post-hypo.

111. A method according to feature 109 or 110 where the safety limits can be set in one or more levels, preferable 3 levels: A work area, a warning area, and an alert area where at least one of the safety limits is broken.

112. A method according to any of the features 109-111 where the at least one safety limit is based or partially based on accumulated insulin delivery over a specific user defined period.

113. A method according to any of the features 109-112, where a safety measure is taken before the actual safety limit is reached, based on the margin between actual blood glucose level and said safety limit and the blood glucose derivative, said derivative being a steep fall or a steep rice in blood glucose level.

114. A method according to any of the features 109-113, where resting/sleep is detected preferable by ECG measurement and the boundary limits are optimized on this basis.

115. A method according to any of the features 109-114 where the user can change modes from the control system normal mode to a safe-mode or completely to manual mode for a period of time.

116. A method according to any of the features 109-115 where a detection of hypoglycemia or a retrospective detection of hypoglycemia is used to change the safety limits.

### DESCRIPTION OF THE DRAWINGS

Embodiments of the invention will now be exemplified with reference to the drawings, in which:

FIGS. 1-3 are insulin and/or blood glucose profiles with corresponding safety limits.

FIG. 4 is a profile representing accumulated flow limit.

FIG. 5 shows different zones of the day and the corresponding insulin flow rate.

FIG. 6 is an overview of safety limits and user defined work area for the closed loop control system.

FIG. 7 is showing limiting of the insulin flow-rate by the user and a control system defined safety limit.

FIG. 8 is showing a 24 hours accumulated limit of insulin amount.

FIGS. 9 and 10 shows possible closed loop control versus manual (passive control system) control during disturbances.

FIG. 11 shows the function of the alarm including user-interaction.

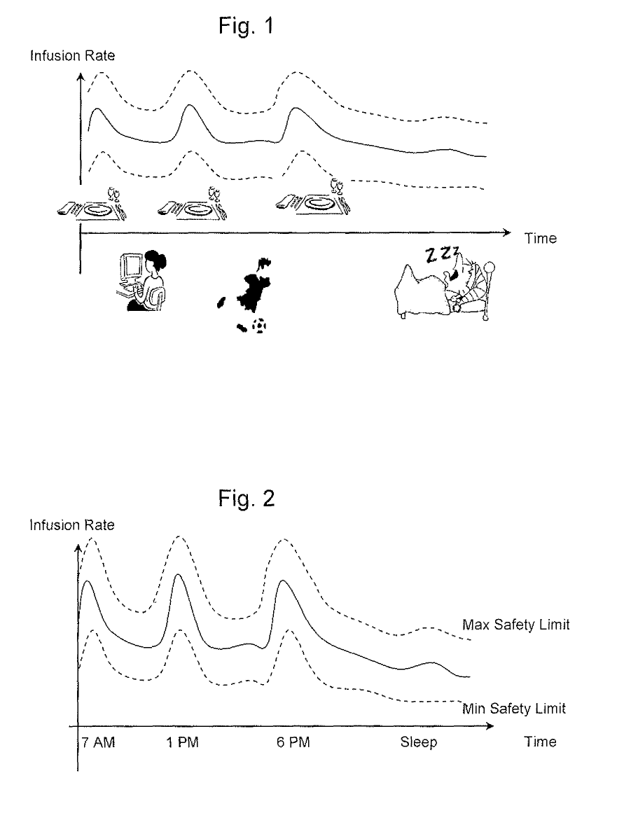

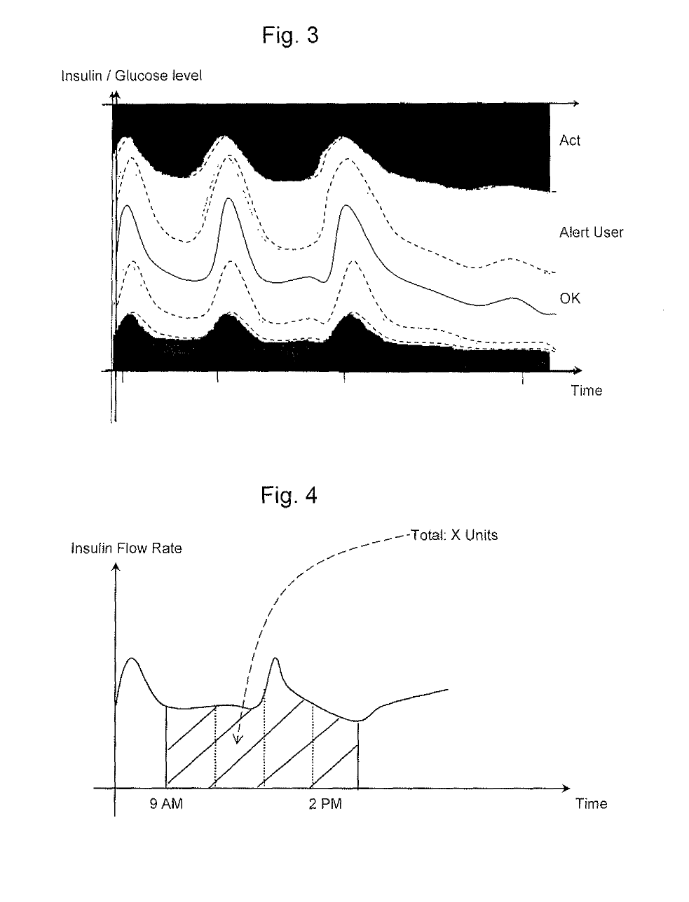

Referring to FIGS. 1, 2 and 3, the safety limits determine different functioning/operating modes which can be divided into three different types:
1. The closed loop algorithm is performing well and can continue (green light).
2. Alert the user (the patient): if a safety limit is broken (yellow light).
3. Limit or stop insulin infusion: if the user does not respond to the alert or the problem becomes severe the system should limit or stop the insulin infusion (red light).

Thus, if the safety limits are not violated the closed-loop control system continues but if a limit is broken the safety system will enter the alert mode (yellow light) and warn the user. Subsequently, five possible actions are present:
A. Reject alarm:
   a. Snooze and forget (i.e. accept break of the limit).
   b. Snooze and learn (i.e. accept break of the limit).
   c. “Autopilot off” (manual government of insulin delivery).
B. Accept alarm (i.e. accept reduced insulin flow).
C. No user-response is provided within a specific time-period and red-light-zone is entered (i.e. reduce or stopped insulin flow).
D. If limit break becomes more severe red-light-zone will be activated.

The setting of safety limits is a method of integrating user-control to a closed loop system of a medical drug here described based on insulin.

The method is based on making a daily disturbance profile being mealtime, size and GI for the meal as well as disturbance based on activity level ranging from sleep to intensive activity.

This prediction of future disturbance is based on historical data, and is used in to inform the user what the safety system predicts will happen the coming minutes and hours (up to 12 hours).

The user can then correct this prediction of future disturbance—etc. If a user normally eat at 1230 but today he is driving in a car and will first eat at 1330—he can then correct the systems prediction and the system will update the prediction according to input.

This way the user and the system can have a knowledge exchange about the disturbance and the result will be a much better control system with optimal insulin dosing and narrow safety limits.

The resulting insulin profile will then be a combination of prior user settings or based on historical data and the correction of future disturbance prediction.

The safety limits as an interval around this estimated insulin-flow profile [see FIGS. 1, 2 and 3]. These limits can be adjusted adaptively.

The key advantage is that the user now has the ability to go from 100% self administration to letting the closed loop system take over gradually. First the user can set the authorization level (safety limits) to say 5% around the prognosis profile. Then as the user has gained trust in the system and has build up knowledge of the performance including system limitations, the system authorization limits can be extended.

Two different sorts of limits are presented. The first is based on a limit of the current insulin infusion rate. The second limits the amount of insulin infusion during a specified period of time (e.g. per 24 hours, night, hour etc.)—i.e. an accumulated limit. An illustration this second limit type is presented on FIG. 4.

Different parameters/features can change the limits. These parameters are described in the following. They may be implemented individually or as a combination of the different schemes.

Historical Normal Flow

By the use of historical insulin infusion an average profile can be determined upon which max and min safety limits can be set. As mentioned above these limits can e.g. be defined and adjusted by the user by setting a percentage interval or an absolute interval. Hence, the safety limits are displaced vertically on the infusion rate axis, and the closed loop control system is limited to infuse insulin at a rate within these boundaries. If the average insulin profile changes, the absolute levels will change accordingly.

Accumulated Limit

Another method of improving the safety of the system is to include a limit for the accumulated/total insulin flow within a specified time period (e.g. per 24 hours, night, hour etc.). As an example this limit can be set in the following way: Accumulated historical data over the last X hours (for the last Y days). The limit is then moving throughout the day but over 24 hours the limit is approximately the same (se FIG. 8).

As an example this can be implemented by calculating an average of the last X hours based on historical data and then setting a max limit of this value.

Limit Based on the Glucose Rate of Change

If steep rise or fall in glucose changes is measured by the CGM the safety system will enter the alert mode and subsequently limit or stop injection if no user-input is provided. E.g. an insulin infusion stop will be caused by steep glucose fall. The total amount of insulin within a five hour period is exemplified in the FIG. 4. This accumulated value is here based on the average insulin profile and must lie within certain boundary limits.

Safety Scheme Based on Activity Measure

Measurement of activity could provide valuable information to the system and displace the safety limits up and down the insulin infusion rate axis. E.g. if high physical activity is detected a safety scheme limits the insulin infusion rate to prevent exercise induced hypoglycemia. (If too low insulin infusion is provided high-intensity exercise can be the cause of intensity-induced hyperglycemia because of counterregulatory hormone response. This results in excessive hepatic glucose output and places a theoretical risk of ketoacidosis.) Another benefit is that the system may not need to “wait” for the CGM to sense decreasing glucose to reduce insulin infusion but immediate action can be taken as the system becomes aware of high physical activity. Thus, activity measurement potentially is able to reduce the problem of delayed glucose sensing in subcutis on steep falling glucose values.

In addition, different kinds of activity influence the present and future insulin sensitivity—and hence the blood glucose—differently (e.g. interval training versus long-distance training). As exemplified in “Perkins & Riddell 2006”. The characterization of exercise into specific exercise groups enables the setting of different safety limits. As the insulin sensitivity may be altered several hours after exercising, this information can be used prospectively as well, thereby reducing the risk of post-exercise hypoglycemia. Furthermore, the activity characterization can also be a parameter in the closed loop control system itself.

Sleep Detection

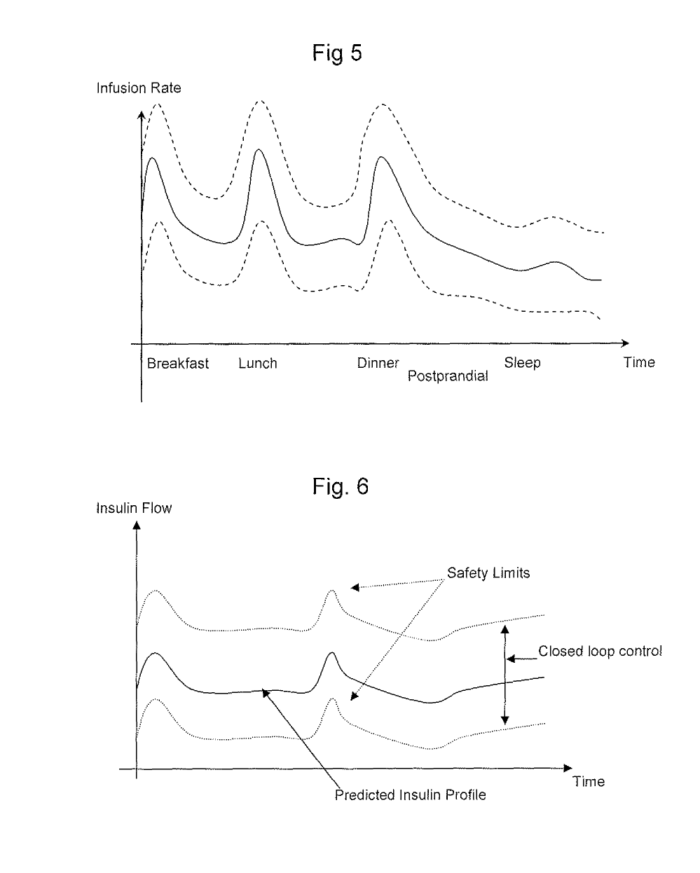

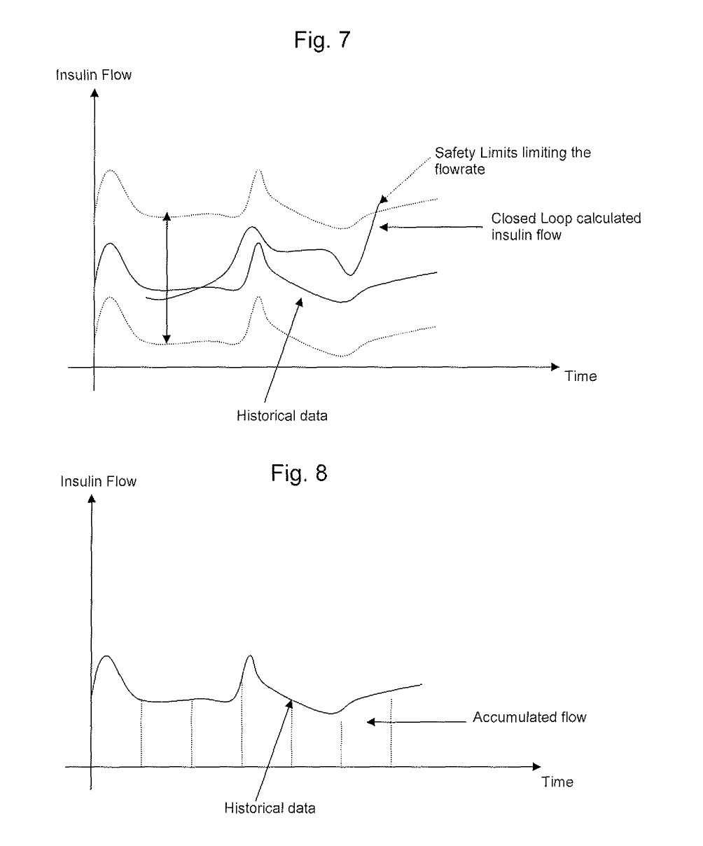

Referring to FIG. 5, another approach based on activity measure is sleep mode detection.

During sleep the conditions of the closed-loop infusion system is changed into a more stable environment because the user is lying down. Sleep can be detected based on ECG and hence optimization of control system settings can be applied during the night—e.g. other safety limits and other control system parameters altering the “aggressiveness” of the control algorithm.

A combination of the activity measurement and sleep detection will change the (limit) settings during the night. If e.g. activity (including categorisation of the exercise type) has been detected during the day the insulin sensitivity will be enhanced during the night increasing the risk of hypoglycemia. Hence, boundary limits of the insulin infusion rate during the night can be changed accordingly. A number of studies in children and adults have demonstrated that most severe hypoglycemic events occur at night and suggest that such events are more frequent after days of increased physical activity. The risk of severe hypoglycemia at night following exercise during the day is a very common concern [The Diabetes Research in Children Network Study Group, 2005]. Thus, the combination between activity measurement and sleep detection will together optimize the algorithm into a nocturnal setting which includes information about daytime activity.

Likewise, the day can be divided into zones that turn the limits/control system into different modes of operation. Thus, as during sleep the limits and the system itself can have different settings according to which zone the user is in. Several zones may be included:
Sleep zone (including “dawn phenomenon mode” if the user experiences this regularly)
Breakfast
Lunch
Postprandial
Regular daytime
Snack
Dinner

FIG. 5 shows different zone of the day and the corresponding insulin flow rate.

Retrospective Hypoglycemia Detection

As a consequence of hypoglycemia a significant elevation of the glucose levels is often seen in the following 6-24 hours. This is known as the rebound phenomenon or Somogyi phenomenon. The phenomenon can be detected retrospectively and provide feedback to the closed loop system by influencing the safety limits as well as the closed-loop control algorithm itself.

Safe-Mode (Autopilot Off)

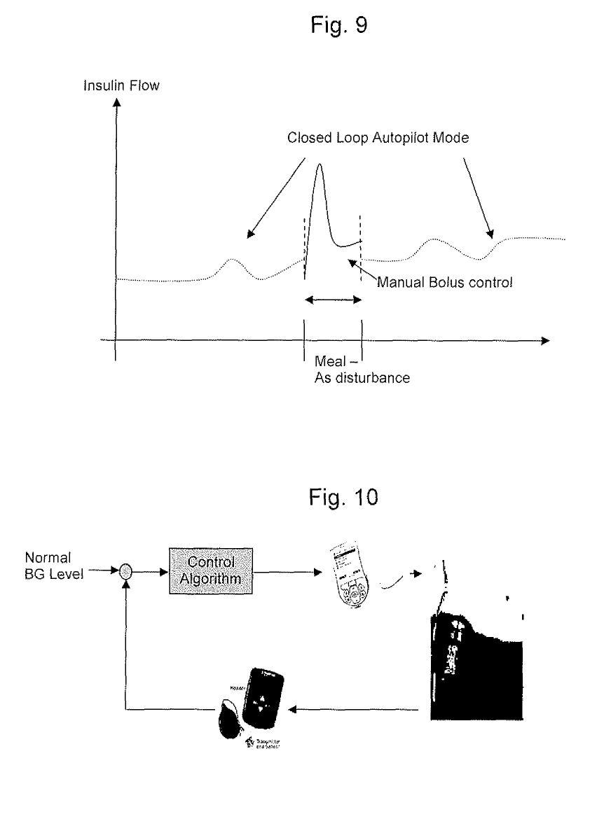

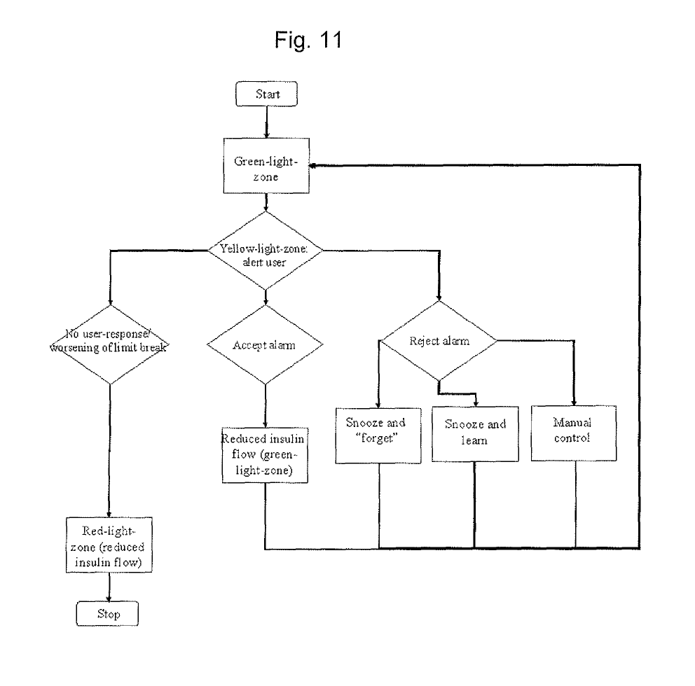

Finally, the closed-loop can be switched off and manual control take over in cases of special/not-learned disturbances like sports, meals etc. which the closed-loop control system is not able to handle. Alternatively, a modest user intervention can switch the infusion into a safe-mode in which only the basal insulin infusion rate is injected. Thus, besides the operating modes green/yellow/red (as previously presented) this introduces an additional mode: “Autopilot off”. “Autopilot off” is the users' ability temporarily to influence the safety limits, i.e. suspend/remove the limits. An example of special disturbance where “autopilot-off” mode is used is provided in FIGS. 9 and 10. In FIG. 11 an overview of how a “meal” (a disturbance) is changing the endogenous balance and the time delay is illustrated.

Hypo-Detection

Detection of hypoglycemia by a hypo-alarm based on ECG and skin impedance is able to overrule the other schemes and will alarm the user. If no user input rejects the alarm, action will be taken by turning off the insulin infusion.

In summary the working area of the insulin pump can be formulated by the following expression:

Safety limits=Expected insulin profile+/−User Autherisation work area in %

The alteration of the safety limits due to influence by the different parameters can be expressed as a combination/weighting of the individual parameters.

### Additional Features:

Learning: The individual algorithms are adaptive and able to be adjusted to the user. In addition, adaptive learning ensures that algorithm settings are adjusted if user changes his/her habits. Besides, if large disturbance is detected the user can tell the system to learn from the event or not. An example of a user-specific event is a bicycle ride on a specific time of the day/week in which sport is made repeatedly. Scenarios that the system should not learn from are “one-time-events” that cause large disturbances in the glucose level.

Control Algorithm in Closed Loop: All the mentioned ways of changing the safety limits of the control system can be implemented in a closed-loop control algorithm itself. E.g. the parameters could be included in a model which predicts the future glucose level.

The major advantage is that the closed-loop control algorithm can be limited to only work within user defined limits based on the average use of insulin from historical data (the last z days). The use of historical data to determine the limits is a simple algorithm and intuitive for the user to handle. The safety schemes provide a tool for reducing the risk of hyperinsulinaemia.

1. A medical device control system for controlling the delivery of insulin into a user comprising:
a blood glucose sensor for sensing at least one physiological parameter,
storing means for storing the at least one physiological parameter and delivery rate over time and occurrences of events as historical data,
processing means to generate a behavioural pattern for the user based on said historical data,
output means for generating an output, said output represents an insulin delivery profile,
audio and/or visual display means for presenting an output to the user,
input means enabling the user to perform manual input to the control system and a medical device in communication with said control system, said processing means via the display means presents an event forecast and a reminder to the user based on said generated behavioural pattern, said event forecast corresponds an insulin delivery rate profile which initiates on the precondition that the user accepts the event forecast,
wherein the control system comprises dynamic safety limits for the insulin profile of the user, whereby said safety limits dynamically surrounds the insulin profile as the insulin delivery level rises and falls, said safety limits thereby has a user defined distance to the insulin profile.

2. A medical device control system according to claim 1 wherein said physiological parameter is the users blood glucose level.

3. A medical device control system according to claim 1, wherein the control system comprises dynamic safety limits for the glucose level of the user, whereby said safety limits dynamically surrounds the profile of the glucose level as the insulin delivery level rises and falls, said safety limits thereby has a user defined distance to the glucose level.

4. A medical device control system according to claim 1, wherein the control system comprises a variable user input default accept threshold whereby event forecasts and their corresponding delivery rate profiles lying within said user input accept threshold are implemented in the control of the delivery device without specific user acceptance of the actual event.

5. A medical device control system according to claim 1, wherein the upper dynamic safety limit is not symmetrically displaced above the insulin profile/the glucose level as compared to the lower dynamic safety limit.

6. A medical device control system according to claim 1, wherein at least one static user set limit correlates the dynamic limit, said static limit is independent of the actual insulin or blood glucose level.

7. A medical device control system according to claim 1, wherein said event forecast and said corresponding insulin delivery rate profile is adaptive, whereby it over time learns to estimate increasingly accurate events and corresponding insulin delivery rate profiles on the basis of the logged historical data, as the base of historical data increases.

8. A medical device control system according to claim 1, wherein the user has the possibility of including or excluding an occurring event from the historical data storage.

9. A method for giving user authorization and limiting a medical device control system; the system controlling the delivery of insulin into a user, said system comprising a sensor for sensing one or more physiological parameter(s) of the user, storing means for storing delivery rate over time and at least one physiological parameter as historical data, processing means to generate a behavioral pattern for the user based on said historical data, output means for generating an output, said output represents an insulin delivery profile, audio and/or visual display means for presenting an output to the user, input means enabling the user to perform manual input to the control system and a medical device in communication with said medical device control system, the method comprising:
Introducing one or more safety limits between which the medical device control system is given user authorization to operate.
setting the safety limit(s) by user input and/or by the control system, based on historical insulin delivery data and at least one further parameter,
dynamically changing at least one safety limit up or down relative to the insulin delivery rate axis or into different event modes, such as high, low, normal and sleeping; based on event monitoring.

10. A method according to claim 9 where the at least one further parameter is: physiological interstitial glucose level or event monitoring, and said event is a meal or postprandial sleep or exercise or post-hypo.

11. A method according to claim 9, wherein the safety limits can be set in one or more levels, preferable 3 levels: A work area, a warning area, and an alert area where at least one of the safety limits is broken.

12. A method according to claim 9, wherein the at least one safety limit is based or partially based on accumulated insulin delivery over a specific user defined period.

13. A method according to claim 9, wherein a safety measure is taken before the actual safety limit is reached, based on the margin between actual blood glucose level and said safety limit and the blood glucose derivative, said derivative being a steep fall or a steep rice in blood glucose level.

14. A method according to claim 9, wherein resting/sleep is detected preferable by ECG measurement and the boundary limits are optimized on this basis.

15. A method according to claim 9, wherein the user can change modes from the control system normal mode to a safe-mode or completely to manual mode for a period of time.

16. A method according to claim 9, wherein a detection of hypoglycemia or a retrospective detection of hypoglycemia is used to change the safety limits.
